(19) United States
(12) Patent Application Publication
(10) Pub. No.: US 2010/0069490 A1
(43) Pub. Date: Mar. 18, 2010

(54) COMPOSITION FOR A PHARMACEUTICAL TREATMENT BASED ON TRIETHYL CITRATE AND ADAPALENE

(76) Inventor: Gianfranco de Paoli Ambrosi, Salo' (Brescia) (IT)

Correspondence Address: MCGLEW & TUTTLE, PC, P.O. BOX 9227, SCARBOROUGH STATION, SCARBOROUGH, NY, 10510-9227, US

(21) Appl. No.: 12/513,555

(22) PCT Filed: Nov. 5, 2007
(86) PCT No.: PCT/IT07/00772

§ 371 Date: May 5, 2009

(30) Foreign Application Priority Data
Nov. 8, 2006 (IT) BS 2006 A 000194

(51) Int. Cl. A61K 31/225; A61P 29/00

(52) U.S. Cl. 514/547

(57) ABSTRACT

The invention proposes a composition for pharmaceutical use containing as active principles triethyl citrate and adapalene in quantities efficacious for use in the treatment of cutaneous pathologies of inflammatory origin, caused by bacterial infections or altered lipid metabolism, both in the treatment of chronologic and photo induced cutaneous ageing,

### FIELD OF THE INVENTION

The objective of this invention is a new composition containing triethyl citrate in combination with adapalene, including the salts and esters, for pharmaceutical use, for oral use in the form of capsules, tablets, syrup, suspension, solutions, powders, granules, microcapsules, micro spheres, Nan spheres, pharmaceutical purposes under controlled release, for parental use, for ocular use, per local use.

### STATE OF THE ART

Acne is a chronic cutaneous disorder involving the pilosebaceous organ and affects about 80% of young adults and adolescents. Acne therapy can represent a challenge in that the response to the different treatments can vary widely depending on the response of the individual and usually requires treatment for an extended period.

If not properly treated, acne can cause the formation of scars whose psychological impact can become an important factor in the quality of life of the subject and jeopardize interpersonal relationships.

The etiology of acne has not been well defined, even though it is commonly considered as a multiform event characterised by abnormal follicular differentiation with hyper keratinisation, increase in the production of sebaceous with an increase in the production of seborrhoea, bacterial colonization and inflammation.

Acne can be considered an inflammatory type of cutaneous pathology that tends to appear during puberty under the influence of androgens. The development of the pathology is usually correlated to an over production of sebum with hyperplasia of the sebaceous gland and hyper keratinisation of the distal part of the hair follicle. The result of this event is the formation of comedo, made up of sebum and keratin. In this situation, a microenvironment able to favour the bacterial colonisation of Propionibacterium acnes is formed. Bacterial colonisation plays a particular role, even if it is not the only one, in the geneses of the inflammatory phase through the release of chemiotaxic factors, cytokines and enzymes, among which lipase.

### The Role of Free Fatty Acids in the Etiopathogeneses of Acne and the Other Inflammatory Forms of the Cutis

The cutaneous surface lipids can be of epidermic or sebaceous origin and in decreasing order of concentration they are composed of: triglycerides and their products of hydrolysis (diglycerides, monoglycerdes and free fatty acids), ester ceros, squalene stearyls, and stearyl esters.

The average composition of the surface lipids for an adult is as follows: triglycerides 41%, wax esters 25%, free fatty acids 16.4%, squalene 12%, glycerides 2.2%, cholesterol esters 2.1%, cholesterol 1.4%.

The cutaneous lipids play an important role in the etiopathogenesis of acne, in particular the free fatty acids. The fatty acids, both esterificated and not, roughly contain the same quantity of saturated and unsaturated fatty acids. The double links of the saturated fatty acids are mainly in position p Δ 6 on Δ 8. These fatty acids are of sebaceous origin.

The free fatty acids cannot be highlighted by the isolated lipids from the sebaceous gland, but can be highlighted in variable quantities on the cutaneous surface. This means that the origin of the free fatty acids on the cutaneous surface is obtained by hydrolysis of the triglycerides. The quantity ratio between triglycerides and free fatty acids is inversely proportional and limited quantities of intermediates of hydrolysis of triglycerides (of glycerides and monoglycerides) are visible on the cutaneous surface level.

A further possible origin of free fatty acids is from hydrolysis of wax esters and esters of cholesterol. It has been proved that hydrolysis of triglycerides is caused by lipase of bacterial origin.

The free fatty acids, in particular those with chain C₁₀ and C₁₂, produce an inflammatory state.

In acne therapy there are two main paths to control the formation of free fatty acids: reduce the production of sebum, using for example antiandrogens such as cyproterone, or reduce the bacterial load, inhibiting therefore the release of lipase, by using antibiotics. Both paths however, may have specific drawbacks. the first has sexual limitations, and in fact cannot be used by the male sex, and the second has a particular limitation in the risk of growing bacterial resistance (a growing phenomena).

### Triethyl Citrate

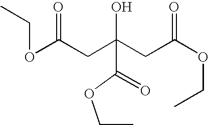

Triethyl citrate is the ethylic ester of citric acid, denomination EINECS: Triethyl-2-hydroxypropan-1,2,3-tricarboxylate, CAS number 201-070-7; minimum formula C₁₂H₂₀O₇; molecular weight 276.29. It is an oily compound soluble in water (up to a concentration of 4.5%) and in lipids.

It has been shown by the Inventor and described for example in document WO2007/066371, that pure Triethyl citrate or in combination with synergisms is able to inhibit hydrolysis of the triglycerides and it acts as a preferential substrate against lipase. From other previous research it has also been established that Triethyl citrate has antibacterial activity (WO2004/019929) and is suitable for cutaneous pathology treatment caused by infections of bacterial origin. Furthermore it has also been shown that Triethyl citrate, in association with ethyl linoleate (WO 03/061766) is able to drastically reduce seborrhoea (up to 68%), to carry out comedolitic activity and to rapidly and efficiently act against different acneic lesions (papules, pustules, cists).

### Adapalene

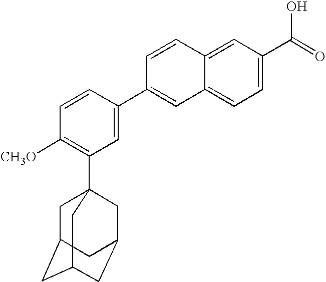

(6-[3-(1-adamantyl)-4-methoxypheny]-2-naphthanoic acid) is a compound similar to retinoids which both in vitro and in vivo, has shown to perform anti-inflammatory activity. The action mechanism of adapalene is correlated to its capacity to bind to specific nuclear receptors of retinoic acid. Applied locally, adapalene exerts a pronounced comedolitic action and performs a marked action against hyper keratinisation and epidermic differentiation performing a normalising effect.

Adapalene modifies the acquired inflammatory and the immune response in acne and, applied locally, it is comedolitic in the rhino mouse model and has effects also on abnormal processes of epidermal keratinisation and differentiation, both present in the pathogeneses of acne vulgaris. The action mechanism of adapalene is proposed in a normalisation of the differentiation of the follicular epithelium cells, which results in a reduced formation of microcomedo.

Adapalene is superior to the reference retinoids in standard anti-inflammatory tests carried out both in vivo and in vitro. Mechanistically, it inhibits the chemiotaxic and chemiokinetic response of the human polymorphonuclear leukocytes and also the metabolism of arachidonic acid directed towards proinflammatory mediators through lipoxidation. This profile suggests that the cellule-mediate inflammatory component present in acne can be modified by adapalene. Studies on human patients supply clinical evidence of the fact that adapalene for cutis application is effective in reducing the inflammatory components of acne (papules and pustules). In document EP 0 199 636 the use of a composition with an adapalene base was proposed in the cure of dermatologic infections and disorders of keratinisation composition which however does not envisage the use of Triethyl citrate.

### OBJECTIVE OF THE INVENTION

This invention was conceived starting from the considerations stated above and on realising that the use of Triethyl citrate in association with adapalene gives even better results in the treatment and cure of etiopathogenesis of acne, seborrhoea, hyper-keratinisation, bacterial colonization and cutaneous inflammation.

Therefore its objective is to supply a pharmaceutical composition containing Triethyl citrate and adapalene as active principles for dermatological treatment to be used preferably for local (in the form of water in oil emulsions, oil in water emulsions, monophase gels, monophase submicelle gels, both aqueous and alcoholic monophase solutions, creams, milks, pomades, ointments) and which can be vehicled in the form of soaked swabs, sprays, etc.

A preferred variation of this invention will be the composition of triethyl citrate and adapalene formulated in the form of an oil in water emulsion, monophase gel, alcoholic lotions and preferably for use in treating seborrhoea and/or acne in the comedon and/or inflammatory phase.

Based on the research carried out by the inventor, the composition based on triethyl citrate and adapalene is able to confer a more marked therapeutic activity and is preferentially designed for the treatment of dermatologic pathologies with inflammatory component chosen between: common acne, comedo, polymorphic acne, nodulocystic acne, conglobate acne, secondary acne induced by solar radiation, by medicine, occupational acne, ichthyosis and ichthyosis forms, Darier syndrome, actinic keratosis, keratosis pilaris, leukoplasia and leuplasiforms, lichen planus, actinic keratosis and actinic lesions of differing origin, rosacea.

The composition containing as active ingredients triethyl citrate and adapalene in combination represent a conceptual and substantial innovation, in particular but not exclusively for acne therapy both of moderate and severe intensity.

### DETAILED DESCRIPTION OF THE INVENTION

In this invention triethyl citrate and adapalene can be used in different concentrations depending on the pharmaceutical form to be used. Triethyl citrate can be used in concentrations varying from 0.05% p/p to 99.0% p/p, preferably from 0.5% p/p to 45% p/p, whereas adapalene can be used in quantities from 0.02% p/p to 2.0% p/p, preferably from 0.1% p/p to 0.5% p/p. EXAMPLES of preparations with triethyl citrate and adapalene base.

PREPARATION 1 - hydro alcohol monophase solution

| N° | Description | % in weight |
|---|---|---|
| 01 | Triethyl citrate | 4.50% |
| 02 | Adapalene | 0.10% |
| 03 | Ethyl Alcohol | 45.0% |
| 04 | water a.r. | 100% |

Method of preparation: dissolve 02) in 03); add 01) to the solution, while stirring add 04) to reach a volume of 100

PREPARATION 2 - gel monophasic

| N° | Description | % in peso |
|---|---|---|
| 01 | Triethyl citrate | 4.5% |
| 02 | Adapalene | 0.20 |
| 03 | Ethyl Alcohol | 10.00 |
| 04 | Carbomer | 1.0% |
| 05 | sodium hydrate a.r. | pH 6 |
| 06 | Distilled water a.r. | 100.00 |

Method of preparation: dissolve 02) in 03), mix 01) to the resulting solution; mix 06) with the resulting solution; disperse 04) in the solution; neutralize with 05). All the operation must be carried out while stirring.

PREPARATION 3 - oil emulsion in water, hydro
soluble triethyl citrate concentration <4.5%)

| | | |
|---|---|---|
| N° | Description | % in weight |

Phase A)

| | | |
|---|---|---|
| 01 | Steareth 2 | 3.0% |
| 02 | Steareth 21 | 2.0% |
| 03 | PPG-15 stearyl ether | 8.0% |
| 04 | Butylhydroxyanisole | 0.01% |

Phase B)

| | | |
|---|---|---|
| 05 | Triethyl citrate | 4.0% |
| 06 | Adapalene | 0.15% |
| 07 | Ethyl Alcohol | 10.00 |
| 08 | Distilled water | 5.0% |

Phase C)

| | | |
|---|---|---|
| 09 | Propylene Glycol | 1.5% |
| 10 | Phenoxyethanol | 0.8% |
| 11 | Methyl parabene sodium salt | 0.25% |
| 12 | Water a.r. | 100% |

Method of preparation: Phase A) mix 01 + 02 + 03 + 04 and heat up to +75° C.; Phase B) dissolve 06 in 07, then mix 05, followed by 0.8; Phase C) dissolve 11 in 12; mix 10 + 09 into the resulting solution and heat up to 75° C.
At 75° C. add Phase A) to Phase C.). Allow to stir in the evening, at 35° C. add Phase B.

PREPARATION 4 - oil emulsion in water, with hydro
soluble triethyl citrate concentration >4.5%

| | | |
|---|---|---|
| N° | Description | % in weight |

Phase A)

| | | |
|---|---|---|
| 01 | Steareth 2 | 3.0% |
| 02 | Steareth 21 | 2.0% |
| 03 | PPG-15 stearyl ether | 8.0% |
| 04 | Butylhydroxyanisole | 0.01% |
| 05 | triethyl citrate | 10.0% |

Phase B)

| | | |
|---|---|---|
| 06 | Adapalene | 0.3% |
| 07 | Ethyl Alcohol | 10.00 |
| 08 | Distilled water | 5.0% |

Phase C)

| | | |
|---|---|---|
| 09 | Propylene Glycol | 1.5% |
| 10 | Phenoxyethanol | 0.8% |
| 11 | Metil parabene sodium salt | 0.25% |
| 12 | Water a.r. | 100% |

Method of preparation: Phase A) mix 01 + 02 + 03 + 04 + 05 and heat to +75° C.; Phase B) dissolve 06 in 07, then mix in 0.8; Phase C) dissolve 11 in 12; mix 10 + 09 to the solution resulting and heat to 75° C.
At 75° C. add Phase A) to Phase C.). Allow to stir in the evening, at 35° C. add Phase B.

PREPARATION 5 - concentrated solution of triethyl citrate

| N° | Description | % in weight |
|---|---|---|
| 01 | triethyl citrate | 40.0% |
| 02 | Adapalene | 0.3% |
| 03 | Ethyl Alcohol a.r. | 100.0% |

Method of preparation: dissolve 02 in 03; mix 01 to the solution.

1. A pharmaceutical composition containing triethyl citrate and adapalene as active principles for use in the treatment of cutaneous pathologies of an inflammatory origin, caused by bacterial infections or by irregular lipid metabolism.

2. A composition according to claim 1, which contains triethyl citrate in a quantity in weight from 0.05% p/p to 99.9% p/p, and adapalene in a quantity from 0.02% p/p to 2% p/p.

3. A composition according to claim 2, which contains triethyl citrate in a quantity in weight preferably from 0.5% p/p to 45% p/p and adapalene in a quantity from 0.1% p/p to 0.5% p/p.

4. A composition according to claim 1, in which said cutaneous pathologies include common acne, comedo, polymorphic acne, nodulocystic acne, conglobate acne, secondary acne induced by solar radiation, by medicine, occupational acne, ichthyosis and ichthyosis forms, Darier syndrome, actinic keratosis, keratosis pilaris, leukoplasia and leuplasiforms, lichen planus, actinic keratosis and actinic lesions of differing origin, rosacea.

5. A pharmaceutical composition containing triethyl citrate and adapalene as active principles for use according to claim 1, for use in the treatment of chronologic and photo induced cutaneous ageing.
